(19) United States
(12) Patent Application Publication
(10) Pub. No.: US 2008/0171931 A1
(43) Pub. Date: Jul. 17, 2008

(54) Device and procedure for cardiac treatment with a MRI - X-ray hybrid system

(76) Inventor: Michael Maschke, Lonnerstadt (DE)

Correspondence Address: BRINKS HOFER GILSON & LIONE, P.O. BOX 10395, CHICAGO, IL, 60610, US

(21) Appl. No.: 11/653,489

(22) Filed: Jan. 16, 2007

(51) Int. Cl. A61B 18/18; A61B 5/055

(52) U.S. Cl. 600/410; 606/41

(57) ABSTRACT

A system and method of treating tachycardias and similar syndromes by the use of catheter ablation of scar tissue is described. The patient is imaged by an X-ray device and an MRI device and the images are fused so as to facilitate identification of scar tissue. The fused image or the X-ray image with treatment areas identified is used to guide the positioning of a catheter with respect to the location to be treated. Guidance of the catheter may be use of X-ray images of the catheter tip, or acoustic or magnetic sensors. After positioning, the catheter is used to ablate body tissue. A further MRI image may be obtained to evaluate the results of the treatment.

### TECHNICAL FIELD

The present application relates to an apparatus and method for treatment of tachycardias, and in particular to using fused imaging technology to facilitate the guidance of a treatment device.

### BACKGROUND

In diseases of the heart that lead to a reduction in the heart rate (bradycardia), cardiac pacemakers that restore the normal sinus rhythm have been used since the 1960s. Other serious cardiological diseases include tachycardial rhythm problems, such as atrial fibrillation. Stimulus-conduction problems in the heart stimulate the atrium at high frequency. In other tachycardias, such as ventricular tachycardias (VTs), complete contraction does not occur, causing defective pumping output of the heart. Classically, the occurrence of tachycardias is reduced by taking medications continuously, or is eliminated by a heart operation in which the stimulus-conduction tissue is severed in certain parts of the heart. This surgical treatment has a relatively high risk for the patient.

VTs originate in the so-called “reentrant circuits”, which are typically created at and in the limit of the electrically nonactive myocardial scar tissue. Recently, a minimally invasive therapy method has become established, where an ablation catheter is introduced via a vein and “burns” the interfering stimulus-conduction paths, for instance with high frequency electrical energy.

A prerequisite for performing ablation therapy is that the problematic stimulus-conduction paths and points be known and be correctly reached with the ablation catheter. For some forms of tachycardia, so-called supraventricular tachycardia, an ablation method can already be called the medical standard. Increasingly, ventricular tachycardias (VTs) are also being treated by this method (see P. Della Bella, “Endocardial Catheter Ablation of Ventricular Tachycardias”), since treatment with medication using antiarrhythmics has a low success rate, and the patient may have to take the medications, which may have substantial side effects, for a long period of time. Implantable defibrillators (ICDs), have unpleasant side effects for the patient. In the case of VTs, however, it is especially difficult during the intervention to identify and reach the sites that have to be “burned out”.

Until now, minimally invasive diagnosis and treatment of tachycardial rhythm problems have been performed with an angiographic X-ray system (see, for example, DE 4436828, “Röntgendiagnostikeinrichtung mit einer Steuervorrichtung für zwei C-Bögen” [“X-Ray Diagnosis System with a Control Device for Two C-Arches”]), a device for recording the intracardial EKG, and a device for “burning out”, or ablation, of the stimulus-conduction problem regions (see, for example, U.S. Pat. No. 5,365,926, “Catheter for Mapping and Ablation and Method Therefore”,) which may be available as a product as the Carto-Mapping system from Biosense Webster, http://wwwjnjgateway.com/). In electrophysiology, this treatment method is generally known as high-frequency ablation or RF ablation. The method for measuring the electrophysiological potentials in the heart for determining the correct ablation site in each case is called mapping.

It would be advantageous, particularly in the case of ventricular tachycardias, if the scar tissue caused by a heart attack in particular could be shown in real time during the procedure for doing ablations in the ventricles, especially the left ventricle.

Magnetic Resonance Imaging (MRI) is especially well suited for displaying scar tissue; see, for example, K. Kim, “Differentiation of Recently Infarcted Myocardium from Chronic Myocardial Scar: The Value of Contrast-Enhanced SSFP-Based Cine MR Imaging”. The MRI provides functional and anatomical imaging. A disadvantage of this approach is that the MRI examination can be done only outside the intervention room, which may be an electrophysiology (EP) laboratory. The MRI examination would have to be performed at a time prior to the electrophysiological procedure, since due to the structural limitations of the gantry, adequate access to the patient does not exist, and the other equipment and instruments would be adversely affected by the magnetic fields of the MRI.

U.S. patent application Ser. No. 11/486,356, “Method and Apparatus for Treating Tachycardial Rhythm Problems”, teaches a way of treating rhythm problems where a display of 3D images of the heart and of the required therapy tools is possible in real time. However, the identification of the ablation points is based solely on the electrical potentials recorded (mapping) in the ventricles of the heart.

An angio-MR apparatus is known (MIYABI from Siemens AG, Munich, Germany), which is a hybrid system comprising MRI and C-arm X-ray system for neurological examinations with a contrast agent (see US Patent Application 2005/0060804, “Support Device of a Patient”). Also a so-called DynaCT (Siemens AG, Munich, Germany); permits computed-tomography-like (CT-like) soft-tissue examinations of biological tissue. A disadvantage of this apparatus is that it is possible only to examine tissue that is not in motion. However, by recording images that are synchronized with an electrocardiogram (EKG) signal, and by subsequent image reconstruction, it is possible to obtain 3D soft-tissue images of the beating heart.

### SUMMARY

A system, method and workflow for treatment of tachycardia is described. In an aspect, the system includes a magnetic resonance imaging modality (MRI) and a C-arm X-ray device, and the MRI image and the CT-like image obtained by processing the X-ray data are fused. The MRI image is used to identify scar tissue in the patient heart, so as to register the scar tissue regions with respect to the CT-like imaging data. The C-arm X-ray system is subsequently used to provide soft tissue imaging so as to assist the operator of an ablation catheter to ablate the scar tissue.

When using a MRI subsystem, with or without administering contrast agents, infarction scars and regions of disturbed or increased electrophysiological activity are identified. The C-arm X-ray subsystem may be used to produce 2D, 3D or 4D (time gated using the EKG data to identify the position in the cardiac cycle) of the anatomy of the heart, including soft-tissue images. The C-arm X-ray images may be obtained either with or without administering X-ray contrast agents. A mixture of these procedures may be performed and images made with or without contrast agents and either superimposed on or subtracted from each other. When the 3D MRI images are fused with the x-ray images, the areas of ventricular scar tissue, for example, can be identified. Securing the X-ray source and detector unit or units to a robot arm with an elbow would be advantageous. This may better access to the patient for administering treatment.

In another aspect, the X-ray source and detector may be attached to one or more robotic arms so as to improve the flexibility of access to the patient and speed in obtain images.

Using the fused images, an ablation catheter may be guided to the desired treatment location by use of images produced by the C-arm X-ray subsystem and related imaging processing and display components. The ablation may be by any technique now known or later developed such as, for example, high frequency (HF) radiofrequency energy, ultrasound, or heat.

In another aspect, a workflow and method for treatment of tachycardia includes: positioning the patient so as to record MRI images, optionally using an injectable contrast agent (such as, for example, gadolinium). Moving the patient to the X-ray system, and obtaining data for producing X-ray images, including soft tissue images, which may be synchronized with the state of the cardiac cycle by EKG data. MRI and X-ray images may be produced, and the images fused using image and data processing techniques, and associated with scar tissue and anatomical aspects of the heart which may be features of the images. Further, other diagnostic data, such as a mapping of electrophysiological data (that is, a map or image or electrical potentials) may also be recorded and superimposed. The areas of the scar tissue or other region to be treated are identified with respect to the X-ray images.

The method and workflow may further include introducing an ablation catheter device into the heart by venous access, for instance, via the aorta and “burning out” or otherwise destroying the regions in the heart that develop unwanted electrophysiological activities. Other access means are possible and improved access methods and apparatus may be used in the future as they become available. The ablation catheter is guided to the treatment site by for example, the fused MRI and X-ray images.

After treatment with the catheter, the patient may be transferred to the MRI subsystem and images obtained to evaluate the results of the treatment. Alternatively, the results may be evaluated using the X-ray subsystem or the electrophysiological subsystem. Depending on the evaluation of the treatment results, the treatment may be concluded, or the imaging and treatment process repeated.

The medical workflow is not limited to treatment of ventricular tachycardias and can be used in analogous manner in ablation treatment of other tachycardias, ventricular flutter and fibrillation as well as for ablation of tumors and metastases.

### BRIEF DESCRIPTION OF THE DRAWINGS

FIG. 1 is a block diagram of a treatment system; and

FIG. 2 is a perspective view of the transfer of a patient between two imaging modalities.

### DETAILED DESCRIPTION

Exemplary embodiments may be better understood with reference to the drawings. Like numbered elements in the same or different drawings perform equivalent functions.

In the interest of clarity, not all the routine features of the examples herein are described. It will of course be appreciated that in the development of any such actual implementation, numerous implementation-specific decisions must be made to achieve a developers' specific goals, such as compliance with system and business related constraints, and that these goals will vary from one implementation to another.

The combination of hardware and software to accomplish the tasks described herein may be termed a system. The instructions for implementing processes of the system may be provided on computer-readable storage media or memories, such as a cache, buffer, RAM, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

The instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a given computer, system, or device.

Where the term “data network”, “web” or “Internet” is used, the intent is to describe an internetworking environment, which may include both local and wide area networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths.

Communications between the devices, the system, subsystems, and applications may be by the use of either wired or wireless connections. Wireless communication may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions.

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

A system for the diagnosis and treatment of, for example, ventricular tachycardia is described. Subsystems may include a magnetic resonance imaging (MRI) subsystem, a C-arm X-ray subsystem and a catheter subsystem. The MRI subsystem may be located near to the remainder of the system; however, portions of the subsystem may be in a separate room so as to avoid the deleterious effects of the magnetic fields on other equipment and objects. The C-arm X-ray subsystem is provided with an X-ray source and an X-ray detector, and may be operated to obtain 2D images, or computed tomography (CT)-like 3D images. The 3D images may be synchronized with the cardiac cycle so as to create 4D images. Apart from the sensors and positioning capabilities, the imaging, data processing and controlling equipment may be located within the treatment room or remotely, and the remotely located equipment may be connected to the treatment room by a communications network. Aspects of the diagnosis and treatment may be performed without personnel except for the patient being present in any of the treatment rooms.

Images obtained by the MRI and the X-ray subsystems may be fused so as to form a composite image by addition, subtraction and the like, including images obtained with and without administering contrast agents. The registration of the images obtained by the imaging modality subsystems may be facilitated by maintaining a known coordinate relationship between the imaging modalities and providing a control of the patient support apparatus that maintains a relationship of the patient control apparatus to the imaging system coordinate systems. For example, the patient may remain on a single stretcher, gurney or the like, and patient support device may be mounted to a dolly or robot so that the patient support apparatus has a known relationship to the physical coordinates of the floor or other surface on which the imaging modalities are mounted. Alternatively the stretcher or gurney may have fudicial structures, such as a pin that engage with a socket on each of the structures that support the stretcher or gurney with respect to the MRI or X-ray systems. Alternative means of registering the images from the two imaging modalities are known and may be employed.

The patient support apparatus may be made of materials that may be substantially transparent to X-rays, and may also be made of materials which are compatible with the MRI apparatus, and may be positionable manually or by a motor or hydraulic mechanism in various coordinate orientations. The patient support apparatus may be mounted to a robot, which may be mounted to a floor, a wall or a ceiling. When the robot is mounted to the floor, the robot may move freely in the horizontal direction, being held in contact with the floor by the force of gravity. The robot may be movable with respect to a surface such as the floor so as to facilitate transferring the patient between treatment stations or rooms, such as between the MRI and X-ray subsystems. The robot may further be capable of transferring the patient to another patient support apparatus, such as an operating table, a bed, or the like. Alternatively, the robot may be guided by rails or the like.

The X-ray imaging modality of the system may further comprise an X-ray tube, high-voltage power supply, radiation aperture, X-ray detector, digital imaging system, system controller, as well as user control and display units. The X-ray detectors may be amorphous Selenium (a-Se), PbI2, CdTe or HgI2 detectors using direct detection and TFT technology, or indirect detectors as is known in the art, or may be subsequently be developed, to provide high resolution, high-dynamic-range real-time X-ray detection. The X-ray source may be rotated around the patient along a circular or elliptical path. The X-ray detector may be disposed diametrically opposed to the X-ray source and such that the plane of the detector is perpendicular to the axis of the X-ray source. This orientation may, for example, be maintained by attaching the X-ray source and X-ray detector to a C-arm, a U-arm or the like. The C-arm may be mounted to a robot so as to permit the X-ray source and detector to be oriented with respect to the patient.

The X-ray imaging device may be operated by rotating, for example, the C-arm such that the opposed X-ray source and X-ray detector traverse an angular range of at least about 180 degrees about an axis perpendicular to the plane of the C-arm. A 3D image may be reconstructed from the detected X-ray data or 2D images may reconstructed in various image planes. For example, a soft tissue image may be reconstructed using the methods described in US Pg-Pub US 2006/0120507 entitled “Angiographic X-ray Diagnostic Device for Rotational Angiography, filed on Nov. 21, 2005”, which is incorporated herein by reference. The algorithmic and measurement aspects of computed tomography images are being improved, and the processing of the images obtained by the imaging devices are expected to continue to improve in resolution and dynamic range, speed, and in reduction of the X-ray dosage.

The term “X-ray” is used to describe any device that uses ionizing radiation to obtain data regarding the opacity of a path through a patient, regardless of the wavelength of the radiation used.

Image quality may be improved by the use of an electrocardiogram (EKG) or respiration-controlled processing of the 2-D projection images used for the synthesis of 3D CT-like images, or for 4D images (that is, time-varying 3D images). One method of using bodily function monitors such as an EKG or respiration monitor is to select the images to be used in the synthesis of a 3D image from portions of the image data set corresponding to similar stages of a heart or respiration cycle. Alternatively, the bodily function monitor may control the movement of the C-arm and the time of obtaining the image data.

The system may include a DICOM (Digital Communication in Medicine) interface including MPPS (Modality Performed Procedure Step), having the capability of further processing the image information and patient data, and interfacing with a data network.

A system or treatment suite may have additional treatment and diagnostic equipment such as a patient monitor, a data terminal for inputting and outputting patient data, such as demographic data, insurance card, laboratory data, patient history and diagnosis information (for example, in the form of a “wireless notebook PC” or the like), various video displays, including projection displays, for displaying data and images, and a digital camera unit for monitoring and video documentation of the individual diagnostic and therapeutic steps. Various signal and data processors may be combined as appropriate with data storage means, displays, control terminals and the like and configured by machine readable instructions to perform the functions and operations described herein.

A robotic arm may facilitate rapid and precise positioning of an imaging device such as the C-arm X-ray subsystem and a robot may be used for positioning of the patient and for moving the patient between the MRI subsystem and the C-arm X-ray subsystem.

The principles of operation of a magnetic resonance imaging (MRI) are known to persons of skill in the art and will not be described in detail. The images produced may be formed in the axial, sagital and coronal planes or an arbitrary plane according the nature of the clinical investigation. Due to the high magnetic fields present in the immediate vicinity of the MRI sensing components, the use of other medical equipment in the near vicinity of the MRI sensing components is limited, and thus the patient is brought to the MRI sensing portion and supported with a patient support apparatus that has been designed to operate in a very high magnetic field (for example, 3 Tesla). After the images of the desired body portion have been obtained, the patient is moved to another area for the X-ray imaging and for treatment.

The MRI images of a heart are useful in identifying differences in the properties of the tissue, and the characteristics of, for example, scar tissue differ from the characteristics of normal tissue. In one aspect of the physiology of tachycardia, the boundary between the scar tissue and the normal tissue has been found to be associated with abnormal electrical potentials that are associated with tachycardia. Ablative removal of the scar tissue in the boundary region is a treatment option for this syndrome.

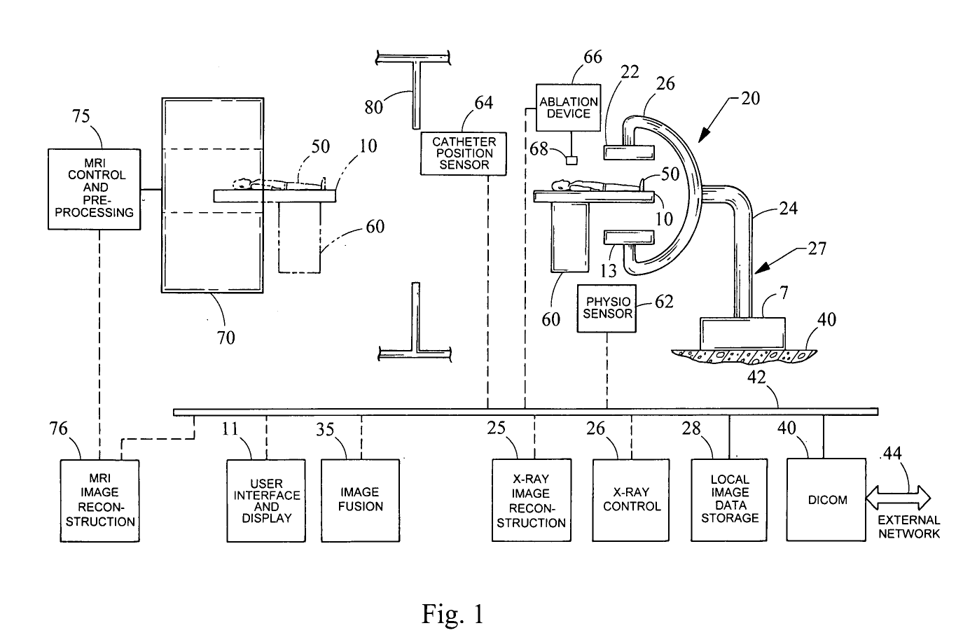

FIG. 1 shows a block diagram of an example of a system for the diagnosis and treatment of an illness by a use of a catheter. Other embodiments of the system may include fewer than all of the devices, or functions, shown in FIG. 1. It will be understood by persons of skill in the art that the signal and data processing and system control is shown in an example, and that many other physical and logical arrangements of components such as computers, signal processors, memories, displays and user interfaces are equally possible to perform the same or similar functions. The particular arrangement shown is convenient for explaining the relationship and functionality of the system.

A C-arm X-ray device 20 is representative of an imaging modality which may be used and comprises a C-arm support 26 to which an X-ray source 22, which may include a diaphragm to limit the field of view, and an X-ray detector 13 may be mounted so as to face each other along a central axis of radiation. The C-arm 26 is mounted to a robotic device 27 comprising a mounting device 7, and one or more arms 24 which are articulated so as to be capable of positioning the C-arm X-ray device with respect to a patient support apparatus 10. The robotic device 27 may be controlled by a control unit 11, which may send commands causing a motive device (not shown) to move the arms 24. The motive device may be a motor or a hydraulic mechanism. The mounting device may be mounted to a floor 40 as shown, to a ceiling or to a wall, and may be capable of moving in longitudinal and transverse directions with respect to the mounting surface.

The C-arm X-ray device 20 is rotatable such that a sequence of projection X-ray images is obtained by an X-ray detector 13 positioned on an opposite side of the patient from the X-ray source 22, and the images are reconstructed by any technique of processing for realizing computed tomographic (CT-like) images. The patient 50 may be positioned on the patient support apparatus 10. The patient support apparatus 10 may be a stretcher, gurney or the like attached to a robot 60. The patient support apparatus 10 may also be attached to a fixed support or adapted to be removably attached to the robot.

The patient may be secured to the patient support apparatus 10 so that the robot 60 may position and reposition the patient during the course of examination, diagnosis or treatment. The attachment of the patient support apparatus 10 to the robot 60 may also serve to maintain the coordinate relationship between the patient 50 and the X-ray apparatus 20 and the magnetic resonance imaging (MRI) apparatus 70. Aspects of the patient support apparatus 10 may be manipulable by the robot 60. Additional, different, or fewer components may be provided.

The devices and functions shown are representative, but not inclusive. The individual units, devices, or functions may communicate with each other over cables or in a wireless manner, and the use of dashed lines of different types for some of the connections in FIG. 1 is intended to suggest that alternative means of connectivity may be used.

The C-arm X-ray radiographic device 20 and the associated image processing 25 may produce angiographic and soft tissue computed tomographic images comparable to, for example, CT equipment, while permitting more convenient access to the patient for ancillary equipment and treatment procedures. A separate processor 25 may be provided for this purpose, or the function may be combined with other processing functions.

Images reconstructed from the X-ray data may be stored in a non-volatile (persistent) storage device 28 for further use. The X-ray device 20 and the image processing attendant thereto may be controlled by a separate controller 26 or the function may be consolidated with the user interface and display 11.

The magnetic resonance imaging device 70 may be located in an adjacent room, separated by a partition 80 from the remainder of the equipment so as to avoid dangerous conditions which may occur if a magnetic material comes too close to the magnets of the MRI device 70. The patient 50 may be moved between the room having the X-ray device 20 and the MRI device 70 while being secured to the patient support apparatus 10, and may also be attached to the robot 60 or other movable support providing that the robot 60 or other support is fabricated out of materials that are compatible with use in a high magnetic field environment. Such as situation is shown by the dashed representation of the patient 50, the patient support apparatus 10 and the robot 60, disposed adjacent to the magnetic resonance imaging device 70.

When operated, the magnetic resonance device 70 produces data which may be used to represent tissue properties in the body of the patient, and may be processed by known image processing techniques so as to provide a volumetric image of a region of interest of the patient 50 by one of a number of means of image reconstruction in a image reconstruction processor 76. The field of image processing is rapidly changing and new and more capable image processing techniques are under constant development. It may be expected that the specific algorithms and techniques used in MRI imaging will continue to improve, as is the case with CT imaging, and that the system may be upgraded so as to use such techniques that are adopted by the medical profession.

The MRI and X-ray images may be obtained with or without various contrast agents that are appropriate to the imaging technology being used, and that the images thus obtained are registered or reconstructed such that the images may be combined into a fused or composite image by image processing techniques such as superposition or subtraction, or the like. This may be performed in a separate image fusion processor 35 or in one of the other system processors.

Additionally, a physiological sensor 62, which may be an electrocardiograph (EKG) a respiration sensor, or the like may be used to monitor the patient 50 so as to enable selection of images that represent a particular portion of a cardiac or respiratory cycle as a means of minimizing motion artifacts in the images.

The treatment device may be an ablation tool 66 having a catheter 68 which is introduced into the body of the patient 50 and guided to the treatment site by images obtained by the C-arm X-ray, or other sensor, such as a catheter position sensor 64. The catheter position sensor may use other than photon radiation, and electromagnetic, magnetic and acoustical position sensors are known.

The various devices may communicate with a DICOM (Digital Communication in Medicine) system 40 and with external devices over a network interface 44.

The X-ray device 20 and the MRI device 70 are located in separate rooms, or otherwise separated for safety purposes. Some or all of the signal and data processing and data display may also be located in the treatment room; however, some or all of the equipment and functionality not directly related to the sensing or manipulating of the patient, may be remotely located. Such remote location is facilitated by high speed data communications on local area networks, wide area networks, and the Internet. The signals representing the data and images may be transmitted by modulation of representations of the data on electromagnetic signals such as light waves, radio waves, or signals propagating on wired connections.

The system sensors, such as the MRI device, physio sensor 62 and X-ray device 20 may thus be located remotely from the specialists making the diagnosis and for determining or administering the appropriate course of treatment. Of course, the specialists may be present with the patient at times as well.

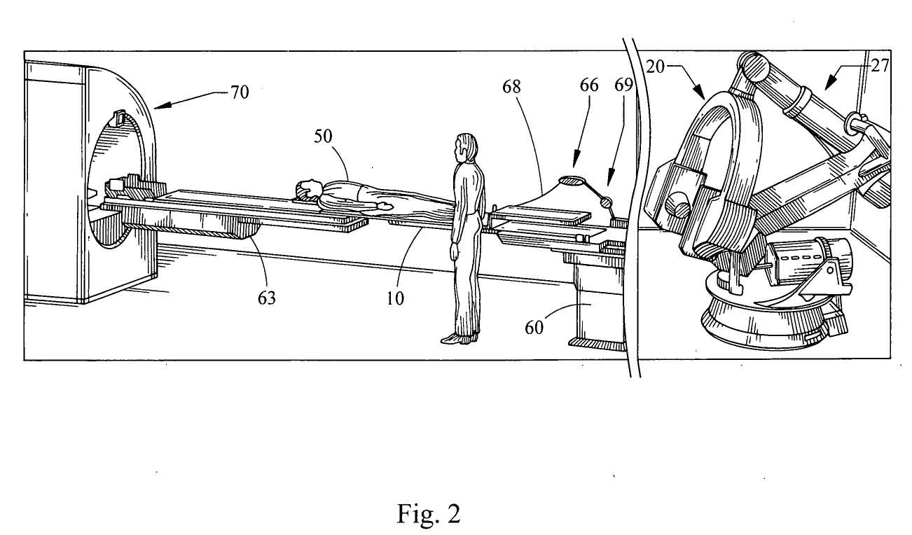

FIG. 2 shows an example where the patient support robot 60 may not be suitable for operation in the strong magnetic field of the MRI device 70. In this case the patient support apparatus 10 is suitable for operation in the MRI magnetic fields and in the X-ray environment. A patient 50 is shown in the process of being transferred from the robot 60 to a patient movement robot 63 associated with the MRI device 70. Typically the robot 63 operates to position the portion of the patient 50 being examined with the aid of the MRI device 70 so that the portion of the patient 50 being thus examined is inside of the MRI device 70, in a particular position with respect to the generated and controlled magnetic fields.

Also shown in an example of an ablation catheter 68 having an ablation device power source 66, and positionable with respect to the patient by robot 69, which may be controlled by using either X-ray or other position sending data which may be displayed with respect to one or more of the fusion images.

A catheter locating system (for example, U.S. Pat. No. 5,042,486, “Catheter Locatable with Non-Ionizing Field and Method for Locating Same”,) for the ablation catheter can be integrated into the system. The catheter may be provided with position sensors, such as electromagnetic sensors or ultrasound-based sensors. Thus the tip of the ablation catheter, in particular, can be detected without emitting continuous X-rays and the motion thereof can be followed and displayed with respect to a previously obtained image.

In another alternative, an Acunav catheter (ultrasound catheter) can be used in addition to the fused MRI and X-ray images, in order to use 3D ultrasound images in real time for guiding the ablation catheter. (see, for example, U.S. Pat. No. 6,923,768, “Method and Apparatus for Acquiring and Displaying a Medical Instrument Introduced into a Cavity Organ of a Patient to be Examined or Treated”).

Some or all of the data collected or processed by the treatment suite may be forwarded to another entity for use in diagnosis, billing and administrative purposes, or further image processing and storage using known interfaces such as DICOM and SOARIAN, or special purpose or later developed data formatting and processing techniques. SOARIAN is a web-browser-based information management system for medical use, integrating clinical, financial, image, and patient management functions and facilitating retrieval and storage of patient information and the performance of analytic tasks (available from Siemens Medical Solutions Health Service Corporation, Malvern, Pa.).

A method of diagnosing or treating a patient is disclosed, including: providing a projection X-ray radiographic apparatus, and providing a MRI imaging apparatus; providing a patient support apparatus; orienting the radiographic apparatus with respect to a patient positioned on the patient support apparatus so as to obtain a sequences of radiographic images, suitable for synthesis of a computed tomography (CT-like) images of a body volume, which may be the heart. The patient is moved to a MRI imaging apparatus and a MRI image of the same or overlapping physical volume is obtained. The images may be obtained with or without the used of contrast agents. Each set of image data is reconstructed so as to yield images which may fused so as to produce a composite image. The sequence of obtaining the MRI and CT-like images may be interchanged.

After the fused images are analyzed by a medical professional so as to identify the specific areas to be treated, the CT-like or the fused image with the areas to be treated is used to assist in the guidance of a treatment device to the treatment site. The guidance may be provided by real-time CT-like images with the treatment site locations superimposed thereon, or by other data such as may be obtained from acoustic or electromagnetic sensing of the catheter position. The catheter may be manipulated by a robot or manually. Once the catheter is guided to the treatment site, the treatment is performed, for example by radio frequency (RF) ablation, or the like. After completion of the treatment, the patient may be transferred to the MRI device and another MRI image obtained to determine if the treatment objectives have been achieved. If the treatment objectives have been met, the procedures is considered completed, but a repeat of the procedure may be necessary if the treatment has not addressed the problem satisfactorily.

Treatment may further include introducing an ablation catheter device into the heart by venous access, for example via the aorta, and “burning out” or otherwise destroying the regions in the heart that develop unwanted electrophysiological activities. The ablation catheter is guided to the treatment site by for example, the fused MRI and X-ray images.

A method of work flow for diagnosis or treatment may therefore include the steps of: positioning the patient with respect to a MRI device so as to obtain imaging data of a volume to be treated; moving the patient from the MRI device to an X-ray subsystem and obtaining imaging data of a corresponding volume. In the process of moving the patient from the MRI device to the X-ray device, the spatial coordinate orientation of the patient with respect to the two devices may be maintained as a system property so that the images obtained by the MRI device and the X-ray device can be fused. The fused images are analyzed to identify artifact to be eliminated. In the case of tachycardia, this is typically the boundary between scar tissue and normal tissue. A catheter is introduced into the patient body and guided to the treatment site using, for example, the X-ray device to provide images of the catheter with respect to the previously obtained X-ray image or the fused image. Alternatively, the catheter may be guided using data obtained by an electromagnetic, magnetic or acoustic sensor, such data being displayed on one or more of the previously obtained or fused images. The catheter is operated so as to treat the selected area. After completing catheter treatment, the patient may again be transferred to the MRI device so as to obtain a confirmatory image. However, should the treatment not have been completed fully, the new MRI image data may be used as new data for fusion with new or existing X-ray data so as to perform additional treatment.

The sequence of steps of obtaining MRI and X-ray data may be altered, and the use of contrast agents and the type of image processing used in the fusion of the images may depend on the treatment protocol. The ablation technique may be used wherever a catheter may be introduced, and may be used to excise or destroy other types of tissue.

At any time during the work flow, a health care professional may choose to modify the sequence of steps, or omit certain steps as the medical circumstances may indicate.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

Although only a few examples of this invention have been described in detail above, those skilled in the art will readily appreciate that many modifications are possible without materially departing from the novel teachings and advantages of the invention. Accordingly, all such modifications are intended to be included within the scope of this invention as defined in the following claims.

What is claimed is:

1. A catheter treatment system, comprising:
an X-ray imaging apparatus;
a magnetic resonance imaging (MRI) apparatus;
a patient support apparatus configured to move a patient secured thereto between examination positions with respect to the X-ray imaging apparatus and the MRI apparatus; and
an image processor configured to fuse images obtained by the X-ray apparatus and the MRI apparatus.

2. The system of claim 1, further comprising a catheter and catheter energy source.

3. The system of claim 1, wherein fused images are transferred to an external data base.

4. The system of claim 1, where the fused images are transferred to an external data base through a DICOM (Digital Communications in Medicine) interface.

5. The system of claim 2, wherein the position of the catheter with respect to the patient is determined by one of the X-ray imaging apparatus, a magnetic sensor, an acoustic sensor or an electromagnetic sensor.

6. The system of claim 1, wherein the selection of data for image reconstruction is controlled using signals obtained from at least one of an electrocardiograph or a respiration sensor.

7. The system of claim 1, wherein data collection for image reconstruction is controlled using signals obtained from one of an electrocardiograph or a respiration sensor.

8. The system of claim 1, wherein the X-ray imaging apparatus is configured to produce computed-tomographic (CT)-like images.

9. The system of claim 8, wherein the CT-like images are soft tissue images.

10. The system of claim 1, wherein the X-ray sensor is manipulated by a first robot.

11. The system of claim 1, wherein the patient support apparatus is moved by a second robot.

12. A method of treating a patient, the method comprising:
placing a patient on a patient support apparatus;
positioning the patient support apparatus with respect to one of a first imaging modality or a second imaging modality;
operating one of the first imaging modality or the second imaging modality to collect data suitable for image reconstruction;
moving the patient to the other of the first imaging modality or the second imaging modality so as to maintain a known orientation of the patient with respect to the first imaging modality and the second imaging modality;
operating the other of the first imaging modality or the second imaging modality to collect data suitable for image reconstruction;
reconstructing images from the first imaging modality and the second imaging modality and fusing the image from the first imaging modality and the second imaging modality so as to form a fused image; and
using the fused image to identify a region of the patient to be treated.

13. The method of claim 12, wherein one of the first imaging modality or the second imaging modality is an X-ray imaging apparatus.

14. The method of claim 13, wherein the other of the first imaging modality or the second imaging modality is a magnetic resonance imaging (MRI) apparatus.

15. The method of claim 13, wherein a contrast agent is administered to the patient in one or more of the steps of operating the X-ray imaging apparatus or operating the MRI apparatus.

16. The method of claim 15, further comprising:
introducing a catheter into the patient;
manipulating the catheter to a region to be treated; and
operating the catheter so as to treat the patient.

17. The method of claim 16, wherein the catheter is operated so as to ablate body tissue.

18. The method of claim 16, wherein the step of manipulating the catheter includes determining a position of the catheter with respect to the patient.

19. The method of claim 18, wherein the step of determining the position of the catheter includes obtaining an image of an X-ray catheter tip.

20. The method of claim 18, wherein the step of determining the position of the catheter includes using an acoustic or a magnetic sensor.

21. The method of claim 18, wherein the patient is moved from the X-ray apparatus to the MRI apparatus and the MRI apparatus is operated to obtain data for image reconstruction, and the treatment concluded or repeated depending on the outcome of treatment previously performed.

22. The method of claim 16, wherein the method further comprises:
manipulating the catheter using a robot.

23. The method of claim 12, wherein the method further comprises:
moving the patient support apparatus between the imaging modalities using a robot.

24. A treatment system, comprising:
means for obtaining soft-tissue X-ray images;
means for obtaining magnetic resonance images;
means for transferring a patient between the means for obtaining soft-tissue X-ray images and the means for obtaining magnetic resonance images while maintaining a known orientation of the patient with respect to each of the means for obtaining the images;
fusing the images obtained from the means for obtaining soft-tissue X-ray images and the means for obtaining magnetic resonance images; and
means for treating a cardiac syndrome.
